(19) United States
(12) Patent Application Publication
(10) Pub. No.: US 2023/0309936 A1
(43) Pub. Date: Oct. 5, 2023

(54) AUTOMATIC MALPOSITION DETECTION OF MEDICAL DEVICES IN MEDICAL IMAGES

(71) Applicant: KONINKLIJKE PHILIPS N.V., EINDHOVEN (NL)

(72) Inventors: AXEL SAALBACH, HAMBURG (DE); ILYAS SIRAZITDINOV, HAMBURG (DE); LEONHARD STEINMEISTER, BIELEFELD (DE); HARALD ITTRICH, HAMBURG (DE); MATTHIAS LENGA, MAINZ (DE); IVO MATTEO BALTRUSCHAT, HAMBURG (DE); MICHAEL GRASS, BUCHHOLZ IN DER NORDHEIDE (DE)

(21) Appl. No.: 18/023,895

(22) PCT Filed: Aug. 26, 2021
(86) PCT No.: PCT/EP2021/073594

§ 371 Date: Feb. 28, 2023

(30) Foreign Application Priority Data
Sep. 2, 2020 (RU) 2020129006

(51) Int. Cl. A61B 6/12; G06T 7/00; G06T 7/73; G16H 50/20; G16H 20/40

(52) U.S. Cl. CPC A61B 6/12; G06T 7/0014; G06T 7/74; G16H 50/20; G16H 20/40; A61M 2025/0166

(57) ABSTRACT

The present invention relates to a system and a method for automatic verification of a positioning of a medical device with respect to an anatomy of a patient in a medical image. A position of a plurality of reference points in a medical image is detected. Further, a presence and a position of a medical device in the medical image is detected. An expected position of the medical device is determined based on the position of the plurality of reference points, and a measure of a correctness of the positioning of the medical device is provided based on a proximity of the position of the medical device to the expected position of the medical device. The measure of the correctness of the positioning of the medical device is provided.

### FIELD OF THE INVENTION

The present invention relates to a system for automatic verification of a positioning of a medical device with respect to an anatomy of a patient in a medical image, and a computer-implemented method for automatic verification of a positioning of a medical device with respect to an anatomy of a patient in a medical image.

### BACKGROUND OF THE INVENTION

Medical images are widespread examinations with multiple possible practical applications and one of the most common examination types in a radiology department. One application case is the detection of malpositioned medical devices. There is a clinical task of detecting and assessing medical device position, since the wrong position of a medical device might not only impact its functionality, but could cause additional complications. For example, the insertion of a central venous catheter (CVC) is notoriously difficult and could result in a pneumothorax. Chest X-ray (CXR), for example, is considered the main modality for assessing the position of central venous catheters, endotracheal tubes, feeding tubes, etc. Thus, after most CVC insertions a chest X-ray is taken to verify the correct position and to check if a pneumothorax is present or not.

Further, chest X-ray is considered to be one of the most common examination in radiology departments due to its relatively low cost and a short acquisition time. As a result, radiology departments may overflow with CXR that may drastically increase report turn-around time. In many studies it was shown, that the report turn-around time for chest X-rays can be up to weeks. The current workflow for the assessment of a chest X-ray, to rule-out a malpositioned CVC and the occurrence of a pneumothorax, involves following steps: A chest X-ray image is acquired. The image of the examination is sent into a PACS system and queued at the end of the worklist. A radiologist examines the chest X-ray after some time x. If a CVC malposition is detected, it is directly reported to a clinician, otherwise normal reporting is done. Then the clinician receives the report after some time y and triggers necessary steps. Both times, x and y, can be long and can add up to several hours or days, which can lead to problems if the CVC is malpositioned in the body and complications may occur.

Hence, there is a clinical need for automatic solutions that can reduce the workload of radiologists in a hospital. Precise and right-time detection of malpositioned medical devices might be very important for particular devices. While generic disease classification or triaging has received tremendous attention, it is notoriously difficult and challenging to reach a sufficient accuracy that would allow for clinical application.

For these reasons, it would be advantageous to have a system and a method for automatic verification of a positioning of a medical device with respect to an anatomy of a patient in a medical image that does not suffer from the above-mentioned drawbacks and provides a reliable malposition detection of medical devices in medical images.

### SUMMARY OF THE INVENTION

It is an object of the present invention to provide a system and a method for automatic verification of a positioning of a medical device with respect to an anatomy of a patient in a medical image that provides that automatically and reliably detects a malposition of medical devices and reports the result to the physician.

The object of the present invention is solved by the subject matter of the independent claims, wherein further embodiments are incorporated in the dependent claims.

The described embodiments similarly pertain to the system for automatic verification of a positioning of a medical device with respect to an anatomy of a patient in a medical image, and the method for automatic verification of a positioning of a medical device with respect to an anatomy of a patient in a medical image Synergistic effects may arise from different combinations of the embodiments although they might not be described in detail.

Further on, it shall be noted that all embodiments of the present invention concerning a method, might be carried out with the order of the steps as described, nevertheless this has not to be the only and essential order of the steps of the method. The herein presented methods can be carried out with another order of the disclosed steps without departing from the respective method embodiment, unless explicitly mentioned to the contrary hereinafter.

According to a first aspect of the invention, there is provided a system for automatic verification of a positioning of a medical device with respect to an anatomy of a patient in a medical image. The system comprises an input unit configured for receiving the medical image comprising at least a part of the patient, and a detection unit configured for detecting a first position of a plurality of reference points of the anatomy of the patient in the medical image, and configured for detecting a presence and a second position of the medical device in the medical image. The system further comprises a determination unit configured for deter wining an expected position of the medical device based on the first position of the plurality of reference points of the anatomy of the patient, and configured for providing a measure of a correctness of the positioning of the medical device based on a proximity of the second position of the medical device to the expected position of the medical device, and an output unit configured for outputting the measure of the correctness of the positioning of the medical device.

Thus, the system allows to automatically check the position of a medical device, as, for example, a central venous catheter. The system comprises a unit for receiving a medical image, which can be an X-ray image like a chest X-ray image. However, also other imagining modalities like magnetic resonance tomography, computed tomography, ultrasound, position emission tomography or SPECT can be employed for acquiring the medical image that is received by the input unit of the system. This medical image depicts at least a part of the anatomy of the patient, like for example the chest. Also, other parts of the body of the patient can be depicted in the image, and also the image can be acquired depicting the whole body. A detecting unit is configured for analyzing the image and for detecting at least one reference point in the image. The position of this reference point is referred to as first position. This reference point can be a certain deter minable point or position of internal organs of the body. One particular suitable reference point for chest X-ray images can be the carina, the location where the trachea divides into the right and left main bronchi. However, any other point can be used as reference point, when the position of this point is determined in the medical image. In most embodiments of the invention, however, a plurality of reference points will be used for detecting their positions, as at least two or three reference point can enable a better determination of the position and orientation of the body and can provide a more reliable positioning of a medical device in the body. The detection unit is further configured for detecting whether a medical device that can be inserted into the body of a patient is present in the body and visible in the image. Particularly in case a medical device is detected in the medical image, the detection unit detects and determines the position of the medical device that is referred to as second position. A determination unit is configured for determining an expected position of the medical device. This expected position corresponds to a correct location and positioning of the medical device within the body and can be defined by the purpose of an examination to be performed within the body, or by the fact that the medical device has not caused a complication during its insertion into the body. The expected position can be determined, for example, by evaluating a position relative to the detected reference points, or by using a statistical model. Further, the determination unit can be configured for comparing the detected second position of the medical device with the expected position. A measure of correctness of the positioning of the medical device is provided. This measure of correctness can be, for example, a probability that the medical device is in a correct position, and/or a deviation of or a proximity to the expected position. Further, the system comprises an output unit that is configured for providing the measure of correctness to a physician, for example. This output unit can comprise a display or at least one light indicating the measure of correctness.

Thus, the system can comprise an anatomical landmark detection module and an object-specific landmark detection module, which can be part of the detection unit. The landmarks correspond to the first position and the second position, respectively. A statistical model of landmark distribution can analyze a spatial correlation between the landmarks and estimate an object position with the surrounding anatomical context. The result of the analysis is reported and can be provided by a visualization module. This can warrant a good interpretability, improve the robustness and enable different modes of visualization and user feedback. The proposed system has the potential to reduce the workload of radiologists, automatically detect critical malpositioned medical objects that constitute a high risk for the patient well-being and may help to prioritize urgent cases. For instance, the position of central venous catheters and endotracheal tubes is usually assessed with respect to the anatomical point carina that navigates the radiologist to the expected region of the correct medical object placement.

In an embodiment of the invention, the detection unit comprises a first artificial intelligence module configured for detecting the first position of the plurality of reference points of the anatomy of the patient and the presence and the second position of the medical device in the medical image.

Implementation of the automatic medical object malposition detection system can start with defining a set of anatomical landmarks for each medical imaging modality and a corresponding set of medical device landmarks, e.g. contour points for pacemakers and centerline points for tubes and catheters. Given the set of anatomical landmarks and the set of object-specific landmarks, a landmark detection model of arbitrary type is built, e.g. classic computer vision model or deep learning model. The model implemented in the detection unit takes a medical image as input and outputs anatomical landmarks and, in case a medical device is detected, also object-specific landmarks. The detection of anatomical landmarks or the first and second position of the reference points and the medical device, respectively can be performed by an artificial intelligence module.

In an embodiment of the invention, the first artificial intelligence module comprises a neural network that is configured for using one of a classification approach, a segmentation technique, a region proposal network, or a pathfinding algorithm.

The artificial intelligence module can comprise a neural network. Further, for the automatic position detection, different embodiments based on neural networks can be considered. In an embodiment, the problem can formulated as a multi-class classification problem to classify the positioning as correct or incorrect. To this end, classification systems such as CNNs can be employed. While a classification approach doesn't provide any information about the position of the medical device, segmentation techniques can be considered. Using techniques such as a UNet, important areas such as the heart, lungs, clavicles, carina as well as the medical device can be identified. After the segmentation, new features can be generated by a relation between the position of the medical device and the surrounding organs to determine a correct position of the medical device. Alternatively, a reinforcement learning algorithm can be employed, where the position of the medical device can be defined as a pathfinding problem. This method needs a starting point on the medical device and follows the medical device towards the end. The starting point can be determined using the anatomy results of the segmentation.

In an embodiment of the invention, the determination unit comprises a probabilistic modelling module configured for determining the expected position of the medical device and/or configured for providing the measure of the correctness of the positioning of the medical device.

A statistical landmark distribution model or probabilistic model can be built in this embodiment of the invention. For that purpose, images can be analyzed where the medical device is placed properly. The statistical model can be implemented as arbitrary probabilistic model, e.g. Markov model, hidden Markov model, deep Bayesian network, etc. There can be two main applications of this statistical model. The first one is to predict the probability of the landmarks of the medical device given the anatomical landmarks. An anomaly score can be provided, which correlates with a probability of the position of the medical device given the position of the reference points. The higher the deviation of a normal and correct positioning is, the higher is the anomaly and/or priority score. The second application is to predict the expected correct position of the medical device given the set of anatomical landmarks, i.e. the first position of the reference points. This application can be used to explain the calculated anomaly and priority scores, and provide a deviation or proximity of the medical device from the expected correct position.

Many modern machine learning and deep learning algorithms can have a major disadvantage of the low interpretability level that makes their usage more difficult in clinical practice. In contrast, the system of the present invention explicitly reflects the relation between the expected object position and the surrounding anatomy. Moreover, it can provide a radiologist with a visualization of where the target object is expected to be placed to explain the calculated anomaly score. Using this approach, the (mal)position of the object can be quantified and automated in a structured reporting, for example outputting that the tip position of a central venous catheter is too high above the carina point by, for example, a vertical distance of 3.0 cm.

In an embodiment of the invention, the probabilistic modelling module comprises a probabilistic model derived from a plurality of datasets comprising medical images of the medical device in a correct position with respect to an anatomy of a patient for determining the expected position of the medical device based on the first position of the plurality of reference points.

In this embodiment of the invention, training or deriving of the probabilistic model can be achieved by analyzing a plurality of datasets with a correct positioned medical device in the body of a plurality of patients. As it is a challenging task to collect cases of malpositioned medical objects for each imaging modality and each medical device, this algorithm can be developed by employing datasets with a correctly positioned medical device. As a result, the proposed system of the present invention overcomes this lack of data problem by learning only on cases with correct positioned medical devices.

In an embodiment of the invention, the system comprises a second artificial intelligence module configured for detecting a complication caused by inserting the medial device into the patient.

Further to the determination of the measure of correctness of the positioning of the medical device, in this embodiment of the invention, the system can comprise a second artificial intelligence module that is configured for detecting complications caused by the insertion of the device. The complication can be, for example, a pneumothorax. This complication can be detected directly by a damage caused by the medical device to the surrounding tissue.

In an embodiment of the invention, the measure of the correctness of the positioning of the medical device comprises a likelihood score and/or the proximity of the second position of the medical device from the expected position of the medical device.

The measure of correctness of the positioning can thus comprise an anomaly score or a likelihood score with a high score if the medical object and the anatomical landmarks are well aligned, i.e. object is well-positioned. In particular, the detected configuration of the object position can be compared to a landmark constellation, which is most probable given the position of the anatomical landmarks. Further, the measure of correctness can comprise the expected position of the medical device, and the computed spatial distances between landmarks of the medical device and, expected landmarks of the medical device based on the anatomical landmarks in order to determine whether the medical device is well-positioned.

According to another aspect of the invention, there is provided an X-ray machine comprising the system for automatic verification of a positioning of a medical device with respect to an anatomy of a patient in a medical image according to any of the preceding embodiments.

The proposed system can run directly on the X-ray machine but is not limited to it. It can be also used for assessment of chest X-ray images in a PACS system. The proposed system according to the invention can be a part of a radiologist worklist prioritization approach. The system according to the invention can also be integrated to existing image acquisition machines to report about high priority cases immediately. The system can be integrated to the PACS system as a reporting algorithm. Furthermore, the information extracted by the system according to the invention can be used to generate a final report thus a radiologist does not have to examine the X-ray image anymore.

In an embodiment of the invention, the system is configured for outputting the measure of the correctness of the positioning of the medical device directly after the medical image is acquired by the X-ray machine.

Thus, the clinician can get a direct feedback after the, for example, chest X-ray is acquired. The final results of the automatic position checking of a medical device can be reported as a smart light feedback were lights and their colors are used to provide a user feedback if the position of the catheter is correct and if no pneumothorax is detected.

According to another aspect of the invention, there is provided a computer-implemented method for automatic verification of a positioning of a medical device with respect to an anatomy of a patient in a medical image. The method comprising the steps of receiving the medical image comprising at least a part of the patient, detecting a first position of a plurality of reference points of the anatomy of the patient in the medical image, and detecting a presence and a second position of the medical device in the medical image. The method comprises further the steps of determining an expected position of the medical device based on the first position of the plurality of reference points of the anatomy of the patient, providing a measure of a correctness of the positioning of the medical device based on a proximity of the second position of the medical device to the expected position of the medical device, and outputting the measure of the correctness of the positioning of the medical device.

The computer-implemented method according to the invention verifies automatically a positioning of a medical device with respect to an anatomy of a patient in a medical image. In a first step, a medical image is received that comprises at least a part of the patient, like for example, the chest. In a second step, a first position of a plurality of reference points of the anatomy of the patient in the medical image is detected. This reference points can be indications of certain clearly visible features of internal organs, like the carina. In a third step, a presence and a second position of the medical device is detected in the medical image. In a fourth step, an expected position of the medical device is determined based on the first position of the plurality of reference points of the anatomy of the patient. In a fifth step, a measure of a correctness of the positioning of the medical device based on a proximity of the second position of the medical device to the expected position of the medical device is provided. In a sixth step, the measure of the correctness of the positioning of the medical device is provided to a user like a physician.

In an embodiment of the invention, the received medical image is a chest X-ray image.

However, also images acquired with different medical imaging modalities can be used according to the invention. In addition, also different parts of the body or even the whole body can be depicted in the acquired image.

In an embodiment of the invention, the medical device inserted into the patient is a catheter or a tube.

The medical device inserted into the patient and to be detected can be a catheter or a tube. This catheters or tubes can be inserted, for example, into veins or into parts of the respiratory system, and guided through the body to their destination, which can correspond to the expected position.

In an embodiment of the invention, the medical device is a central venous catheter and the correctness of the positioning is defined in that the medical device has not caused a pneumothorax during inserting the medial device into the patient and is in the correct position within the body.

In this embodiment, the medical device is a central venous catheter that can be inserted into one of the internal jugular veins or into one of the subclavian veins and placed near the carina. As there is a risk that the catheter causes complications like a pneumothorax during this procedure, the positioning of the catheter with respect to the anatomy of the patient can be automatically verified according to this embodiment of the invention.

According to another aspect of the invention, there is provided a computer program element, which, when executed on a processing unit, instructs the processing unit to perform the computer-implemented method according to any of the preceding embodiments.

The computer program element can be perform on one or more processing units, which are instructed to perform the method for automatic verification of a positioning of a medical device with respect to an anatomy of a patient in a medical image.

According to another aspect of the invention, there is provided a processing unit configured for executing the computer program element according to the preceding embodiment.

The processing unit can be distributed over one or more different devices executing the computer program element according to the invention. Thus, the benefits provided by any of the above aspects equally apply to all of the other aspects and vice versa.

In a gist, the invention relates to a system and a method for automatic verification of a positioning of a medical device with respect to an anatomy of a patient in a medical image A position of a plurality of reference points in a medical image is detected. Further, a presence and a position of a medical device in the medical image is detected. An expected position of the medical device is determined based on the position of the plurality of reference points, and a measure of a correctness of the positioning of the medical device is provided based on a proximity of the position of the medical device to the expected position of the medical device. The measure of the correctness of the positioning of the medical device is provided.

The above aspects and embodiments will become apparent from and be elucidated with reference to the exemplary embodiments described hereinafter. Exemplary embodiments of the invention will be described in the following with reference to the following drawings:

### BRIEF DESCRIPTION OF THE DRAWINGS

FIG. 1 shows a schematic set-up of a system for automatic verification of a positioning of a medical device with respect to an anatomy of a patient in a medical image.

FIG. 2 shows a schematic set-up of an X-ray machine comprising the system for automatic verification of a positioning of a medical device with respect to an anatomy of a patient in a medical image.

FIG. 3 shows a medical image with detected first positions of reference points and second positions of a medical device.

FIG. 4 shows a block diagram of a computer-implemented method for automatic verification of a positioning of a medical device with respect to an anatomy of a patient in a medical image.

### DETAILED DESCRIPTION OF EMBODIMENTS

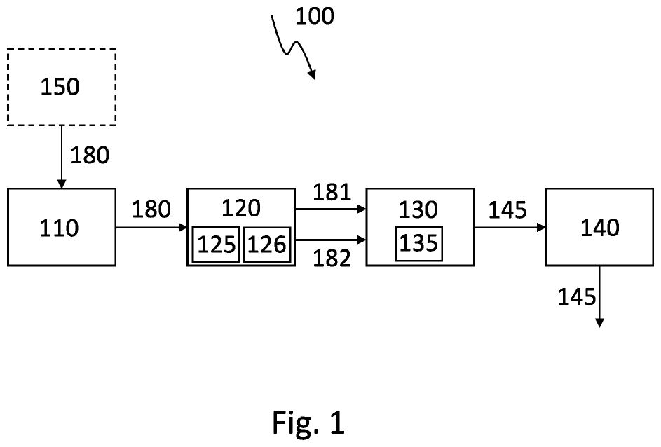

FIG. 1 shows a schematic set-up of a system 100 for automatic verification of a positioning of a medical device 170 with respect to an anatomy of a patient 160 in a medical image 180. The medical image 180 is acquired by an image acquisition unit 150 that may be part of an X-ray machine, and is received by the input unit 110 of the system 100. The detection unit 120 receives the medical image 180 and detects the first position 181 of the plurality of reference points in the medical image 180. Further, the detection unit 120 can detect a presence and a second position 182 of the medical device 170 in the medical image 180. This can be achieved by means of n first artificial intelligence module 125 that can be comprised in the detection unit 120. The detection unit 120 can further comprise a second artificial intelligence module 126 configured for detecting a complication in the medical image 180 caused by the medical device 170. The first position 181 and the second position 182 are provided to the determination unit 130, which can comprise a probabilistic modelling module 135. The determination unit 130 is configured for determining an expected position of the medical device 170 based on the first position 181 of the plurality of reference points of the anatomy of the patient 160, and configured for providing a measure of a correctness 145 of the positioning of the medical device 170 based on a proximity of the second position 182 of the medical device 170 to the expected position of the medical device 170. The measure of correctness can comprise, for example, a degree of deviation of the second position 182 of the medical device 170 from the expected position, a probability for a correct positioning, or the deviation from the expected correct position. The output unit 140 provides the measure of correctness 145 to, for example, a physician. This output can comprise a sign that the medical device 170 is in the correct position, or, for example, that the medical device 170 is located at a certain distance in a certain direction with respect to a first position 181 of a specific reference point.

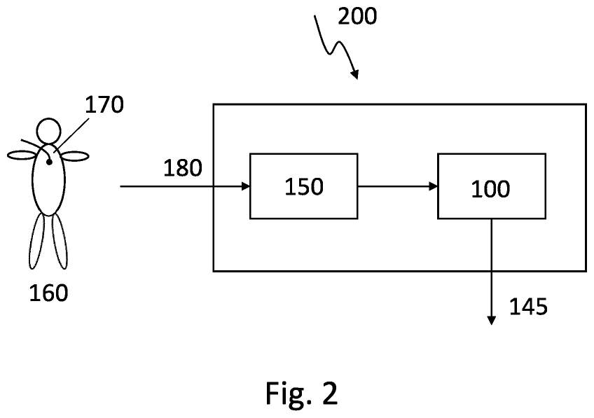

FIG. 2 shows a schematic set-up of an X-ray machine 200 comprising the system 100 for automatic verification of a positioning of a medical device 170 with respect to an anatomy of a patient 160 in a medical image 180. The X-ray machine 200 can further comprise an image acquisition device 150. The image acquisition device 150 of the X-ray machine 200 acquires a medical image 180 of at least a part of a patient 160. In the patient 160, a medical device 170 like a catheter can be inserted. The system 100 analyses the medical image 180 and provides the measure of correctness 145 as output via the output unit 140.

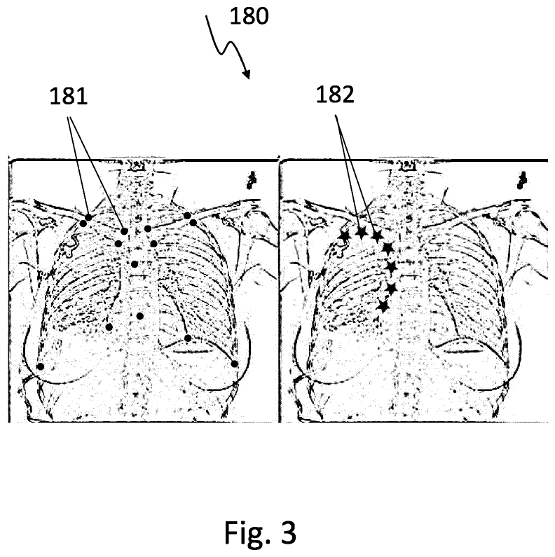

FIG. 3 shows a medical image 180 with detected first positions 181 of reference points and second positions 182 of a medical device 170. The input medical image 180 can be acquired by any arbitrary imaging modality. The detection unit 120 detects the first position 181 of a plurality of reference points as a set of anatomical landmarks, and the second position 182 of a medical device 170 as device specific landmark. In a training phase of the probabilistic model, a statistical model of the landmarks distribution can be derived resulting in a landmarks distribution model. From this model, the expected position of the medical device 170 can be derived. By comparing the expected position of the medical device 170 with the detected second position 182 of the medical device 170, the anomality score can be calculated and a measure of correctness 145 of the positioning of the medical device 170 inside the patient 160 can be returned.

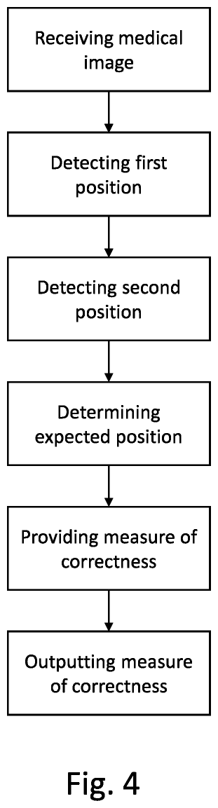

FIG. 4 shows a block diagram of a computer-implemented method for automatic verification of a positioning of a medical device 170 with respect to an anatomy of a patient 160 in a medical image 180. The method comprises a first step of receiving the medical image 180 comprising at least a part of the patient 160. This step is followed by a second step of detecting a first position 181 of a plurality of reference points of the anatomy of the patient 160 in the medical image 180. In a third step, a presence and a second position 182 of the medical device 170 in the medical image 180 is detected. In a fourth step, an expected position of the medical device 170 based on the first position 181 of the plurality of reference points of the anatomy of the patient 160 is determined. This step is followed by a fifth step of providing a measure of a correctness 145 of the positioning of the medical device 170 based on a proximity of the second position 182 of the medical device 170 to the expected position of the medical device 170. In a sixth step, an output of the measure of the correctness 145 of the positioning of the medical device 170 is provided.

In one specific implementation the actual landmark positions can be described in terms of a (translation invariant) configuration vector, as is described in detail in T. V. Pham and A. W. M. Smeulders, “Object recognition with uncertain geometry and uncertain part detection,” Computer vision and image understanding, vol. 99, no. 2, pp. 241-258, 2005:

z*=(x₂−x₁, . . . ,xₚ−x₁,y₂, . . . ,yₚ−y₁)^{T},

where xₙ, and yₙ relate to coordinates of the landmarks reference points respective the detected medical devices. Under the assumption of a multi-dimensional Gaussian, the probability of a configuration can be estimated via:

p(z*)=N₂ₚ₋₂(μ,Σ).

For missing landmarks, also a conditional probability distribution can be considered:

N(θ₁+Σ₁₂Σ₂₂⁻¹(t₂−θ₂),Σ₁₋₂),

with

$$Θ = ( \begin{matrix}{Θ}_{1} \\ {Θ}_{2}\end{matrix} ) and Σ = ( \begin{matrix}{Σ}_{11} & {Σ}_{12} \\ {Σ}_{12}^{⊤} & {Σ}_{22}\end{matrix} )$$

and

Σ₁₋₂=Σ₁₁−Σ₁₂Σ₂₂⁻¹Σ₁₂^{T},

While the invention has been illustrated and described in detail in the drawings and foregoing description, such illustration and description are to be considered illustrative or exemplary and not restrictive. The invention is not limited to the disclosed embodiments. Other variations to the disclosed embodiments can be understood and effected by those skilled in the art in practicing a claimed invention, from a study of the drawings, the disclosure, and the dependent claims.

In the claims, the word “comprising” does not exclude other elements or steps, and the indefinite article “a” or “an” does not exclude a plurality. The mere fact that certain measures are re-cited in mutually different dependent claims does not indicate that a combination of these measures cannot be used to advantage. Any reference signs in the claims should not be construed as limiting the scope.

### LIST OF REFERENCE SIGNS

100 system
110 input unit
120 detection unit
125 first artificial intelligence module
126 second artificial intelligence module
130 determination unit
135 probabilistic modelling module
140 output unit
145 measure of correctness
150 image acquisition device
160 patient
170 medical device
180 medical image
181 first position
182 second position
200 X-ray machine

1. A system for automatic verification of a positioning of a medical device with respect to an anatomy of a patient in a medical image, the system comprising:
an input configured for receiving the medical image comprising at least a part of the patient;
a detector configured for detecting a first position of a plurality of reference points of the anatomy of the patient in the medical image, and configured for detecting a presence and a second position of the medical device in the medical image;
a determiner configured for determining an expected position of the medical device based on the first position of the plurality of reference points of the anatomy of the patient, and configured for providing a measure of a correctness of the positioning of the medical device based on a proximity of the second position of the medical device to the expected position of the medical device; and
an output configured for outputting the measure of the correctness of the positioning of the medical device.

2. The system according to claim 1, wherein the detector comprises a first artificial intelligence module configured for detecting the first position of the plurality of reference points of the anatomy of the patient, and for detecting the presence and the second position of the medical device in the medical image.

3. The system according to claim 2, wherein the first artificial intelligence module comprises a neural network that is configured for using one of a classification approach, a segmentation technique, a region proposal network, and a pathfinding algorithm.

4. The system according to claim 1, wherein the determiner comprises a probabilistic modelling module configured for determining the expected position of the medical device and/or configured for providing the measure of the correctness of the positioning of the medical device.

5. The system according to claim 4, wherein the probabilistic modelling module comprises a probabilistic model derived from a plurality of datasets comprising medical images of the medical device in a correct position with respect to an anatomy of a patient for determining the expected position of the medical device based on the first position of the plurality of reference points.

6. The system according to claim 1, wherein the system comprises a second artificial intelligence module configured for detecting a complication caused by inserting the medial device into the patient.

7. The system according to claim 1, wherein the measure of the correctness of the positioning of the medical device comprises a likelihood score and/or the proximity of the second position of the medical device from the expected position of the medical device.

8. (canceled)

9. (canceled)

10. A computer-implemented method for automatic verification of a positioning of a medical device with respect to an anatomy of a patient in a medical image, the method comprising:
receiving the medical image comprising at least a part of the patient;
detecting a first position of a plurality of reference points of the anatomy of the patient in the medical image;
detecting a presence and a second position of the medical device in the medical image;
determining an expected position of the medical device based on the first position of the plurality of reference points of the anatomy of the patient;
providing a measure of a correctness of the positioning of the medical device based on a proximity of the second position of the medical device to the expected position of the medical device; and
outputting the measure of the correctness of the positioning of the medical device.

11. The computer-implemented method according to claim 10, wherein the received medical image is a chest X-ray image.

12. The computer-implemented method according to claim 10, wherein the medical device inserted into the patient is a catheter or a tube.

13. The computer-implemented method according to claim 10, wherein the medical device is a central venous catheter and the correctness of the positioning is defined in that the medical device has not caused a pneumothorax during inserting the medial device into the patient.

14. (canceled)

15. (canceled)

16. The computer-implemented method according to claim 10, further comprising providing a first artificial intelligence module configured for detecting the first position of the plurality of reference points of the anatomy of the patient, and for detecting the presence and the second position of the medical device in the medical image.

17. The computer-implemented method according to claim 16, wherein the first artificial intelligence module comprises a neural network that is configured for using one of a classification approach, a segmentation technique, a region proposal network, and a pathfinding algorithm.

18. The computer-implemented method according to claim 10, further comprising a probabilistic modelling module configured for determining the expected position of the medical device and/or configured for providing the measure of the correctness of the positioning of the medical device.

19. The computer-implemented method according to claim 18, wherein the probabilistic modelling module comprises a probabilistic model derived from a plurality of datasets comprising medical images of the medical device in a correct position with respect to an anatomy of a patient for determining the expected position of the medical device based on the first position of the plurality of reference points.

20. The computer-implemented method according to claim 10, further comprising providing a second artificial intelligence module configured for detecting a complication caused by inserting the medial device into the patient.

21. The computer-implemented method according to claim 10, wherein the measure of the correctness of the positioning of the medical device comprises a likelihood score and/or the proximity of the second position of the medical device from the expected position of the medical device.
